(19) United States
(12) Patent Application Publication
(10) Pub. No.: US 2007/0148209 A1
(43) Pub. Date: Jun. 28, 2007

(54) Allergic constitution ameliorator

(75) Inventors: Michihiro Hide, Hiroshima (JP); Yoshikazu Kameyoshi, Hiroshima (JP); Hidenori Suzuki, Hiroshima (JP); Souichi Oomizu, Hiroshima (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: NISHIKAWA RUBBER CO., LTD., Hiroshima-shi (JP)

(21) Appl. No.: 10/578,914

(22) PCT Filed: Nov. 10, 2004
(86) PCT No.: PCT/JP04/16676

§ 371 Date: May 9, 2006

(30) Foreign Application Priority Data
Nov. 11, 2003 (JP) 2003-381557

(51) Int. Cl. A61K 47/00; A61K 31/715

(52) U.S. Cl. 424/439; 514/54

(57) ABSTRACT

An agent for improving allergic predisposition is provided. This agent is effective in improving the allergic predisposition, safe, and readily ingestible. More specifically, an agent for improving allergic predisposition and an agent for suppressing production of IgE antibody containing a hydrolysate of water-soluble dietary fibers as its effective component are provided.

### TECHNICAL FIELD

This invention relates to an agent for improving allergic predisposition which has an action of suppressing production of IgE antibody. This agent is effective in improving the allergic predisposition.

### BACKGROUND ART

People with allergic predisposition susceptible to allergic diseases such as pollinosis, allergic rhinitis, bronchial asthma, atopic dermatitis, and drug eruption are increasing particularly in advanced industrial countries due to increase of stress caused by environmental deterioration, change in the life style, and complication of social life, and there is a strong demand for a countermeasure that can be used in improving the allergic predisposition.

Allergy is a disease caused by abnormal hypersensitivity of immune system that results in self attacking of one's own tissue although immune system is originally a defense system for non-self attacking outer enemies (foreign substances). There are 4 types of allergies. Type I to type III allergies are all caused by an antibody. Among these, type I allergy is called immediate allergy since symptoms appear as early as 2 to 3 minutes after the contact with the antigen and the peak comes in less than 20 minutes. Type IV allergy is called delayed allergy since this lymphocytes are involved in this type of allergy and the symptoms gradually appear in 24 to 48 hours after the invasion of the antigen into the body. Typical type I allergies include atopic dermatitis, acute urticaria, bronchial asthma, pollinosis, rhinitis, and gasterointestinal allergy, and this type is the most frequent type of the allergy. Type II allergies include hemolytic anemia, decrease of platelet, blood type incompatibility, allergy to drugs; type III allergy include serum sickness, glomerulonephritis, chronic pneumonitis, chronic articular rheumatism, and viral hepatitis; and type IV allergies include contact dermatitis, tuberculosis, rejection to organ to the transplant, and allergy to metals.

The onset mechanism of the type I allergy involving the IgE antibody is thought to be as described below. When any of various foreign antigens such as food antigen like egg, milk, or soybean and inhaled antigen such as mite or pollen invades the body, and the antigen that has become incorporated in the antigen presenting cell is presented to CD4+ T (helper T) cell, the helper T cell which has recognized the antigen presented by the antigen presenting cell reacts with B cell to cause differentiation and propagation of the B cell into an antibody forming cell.

There are two types of helper T cells depending on the type of the cytokine produced: type I helper T cell (Th1 cell) which mainly induce cell-mediated immunity, and type II helper T cell (Th2 cell) which induce humoral immunity (See, for example, Non-patent Document 1). Typical cytokines produced by the Th1 cell include interleukin 2 (IL-2), interferon γ (IFN-γ), and TGF-β, and typical cytokines produced by the Th2 cell include IL-4, IL-5, IL-6, IL-10, and IL-13. Naive T cell not sensitized by the antigen is known to differentiate into Th1 cell by the acting with antigen presenting cell and IL-12, and into Th2 cell by acting with antigen presenting cell and IL-4.

By an interaction with the Th2 cell associated with allergic reaction, B cell matures into an antibody-forming cell, and as it propagates, produces IgM antibody, and then, IgG1 antibody, and finally, IgE antibody. In the meanwhile, the B cell which reacted with the Th1 cell grows into an antibody-forming cell, and as it propagates, produces IgM antibody, IgG2a antibody, IgG2b antibody, and IgG3 antibody, while the most important function of the Th1 cell is activation of macrophage by the action of the IFN-γ produced by the Th1 cell to thereby induce cell-mediated immunity.

The thus produced IgE antibody binds to the surface of the mast cell, and when the specific antigen binds to the IgE antibody, the mast cell is activated to release inflammatory substance such as histamine to induce the allergic condition. The chemotactic factor released from the mast cell attracts eosinophil having inflammatory action to worsen the inflammation, and the interleukin 4 (IL-4) and IL-13 further promote the IgE antibody production by the B cell.

Th1 cell and Th2 cell mutually suppress each other's action. For example, IFN-γ produced by the Th1 cell is known to suppress the IgE antibody production. In the healthy condition, the balance between the Th1 and Th2 cells are maintained, and the IgE antibody is not produced against the invading antigen. In contrast, in the allergy patient, the balance between the Th1 and Th2 cells is disturbed and the Th2 cell is superior to the Th1 cell in responding to the invading antigen. The patient, therefore, is prone to suffer from the allergy involving IgE antibody (see, for example, Non-patent Document 2).

In the prevention or therapy of an allergic disease, a drug such as steroid, antihistaminic agent, chemical mediator release inhibitory agent, or immunosuppressive has been used. It has also been reported that glucomannan or galactomannan that has been hydrolyzed has the effect of reducing intake of allergen or microorganism in the intestine (see for example, Patent Document 1). While such mechanisms may suppress onset of allergic conditions or alleviate the allergic conditions, allergic predisposition itself is not improved by such mechanism.

In contrast an agent which is capable of suppressing the production of the IgE antibody is expected to improve the allergic predisposition, and compounds such as suplatast tosilate and strictinin have been found. It has also been reported that glucomannan and its pulverizate have action of suppressing the IgE antibody (see, for example, Patent Document 2).

However, a compound such as suplatast tosilate is associated with the problem of side effects, and the glucomannan had the problems of insufficient effectiveness, requirement for specialized equipment and technique of pulverization, excessively long time until the manifestation of the action, and limited dosage form due to unsuitability for preparation into an injection.

[Patent Document 1] JP-A-2003-513893

[Patent Document 2] JP-A-2003-55233

[Non-patent Document 1] Mosmann, T. R. et al., J. Immunol., 136, 2348-2357, 1986

[Non-patent Document 2] “Standard Immunology” edited by Taniguchi, K and Miyasaka M., published from Igaku-Shoin, 2001

### SUMMARY OF THE INVENTION

An object of the present invention is to provide an agent for improving allergic predisposition which is effective in improving the allergic predisposition, and is safe, and readily ingestible.

The inventors of the present invention conducted an intensive study by focusing on the anti-allergic action of dietary fibers, and found that polysaccharides produced by hydrolyzing water soluble dietary fibers have excellent action of suppressing IgE antibody production and are well adapted for administration, and therefore, such hydrolysate can be used as a drug and food effective in improving the allergic predisposition. The present invention has been completed on the bases of such finding.

Accordingly, this invention provides an agent for improving allergic predisposition containing a hydrolysate of water-soluble dietary fibers as its effective component.

This invention also provides an agent for suppressing production of IgE antibody containing a hydrolysate of water-soluble dietary fibers as its effective component.

This invention also provides a food for improving allergic predisposition containing hydrolysate of water-soluble dietary fiber.

This invention also provides a use of a hydrolysate of water-soluble dietary fibers for improving allergic predisposition.

This invention also provides a use of a hydrolysate of water-soluble dietary fibers for producing an agent for suppressing production of IgE antibody

This invention also provides a method for improving allergic predisposition comprising administering a hydrolysate of water-soluble dietary fiber.

This invention also provides a method for suppressing production of IgE antibody comprising administering a hydrolysate of water-soluble dietary fiber.

The hydrolysate of the water soluble dietary fiber of the present invention is effective in improving allergic predisposition of a person who is susceptible to type I allergy such as atopic dermatitis since it has action of suppressing the IgE antibody production. The hydrolysate of the water soluble dietary fiber of the present invention is also suitable for administration as a peroral preparation, and also, as a parenteral preparation such as injection preparation because of its low molecular weight and high solubility. In addition, since dietary fiber is routinely ingested, the product of the present invention has extremely low risk in terms of safety, and can be used as a safe, readily ingestible food for people of all ages from infant to the elderly.

### BRIEF DESCRIPTION OF THE DRAWINGS

FIG. 1 is a graph showing the effect of suppressing IgE antibody production by hydrolysate of konjac glucomannan.

FIG. 2 is a graph showing the effect of suppressing IgE antibody production by the hydrolysate of galactomannan.

FIG. 3 shows the results of gel filtration chromatography of the hydrolysates of konjac glucomannan produced by using hydrochloric acid at different concentration.

FIG. 4 is a graph showing the effect of suppressing the IgE antibody production by the hydrolysate of konjac glucomannan produced by using hydrochloric acid of different concentration for the hydrolysis.

FIG. 5 is a graph showing the effect of the hydrolysate of konjac glucomannan on suppressing the IgE antibody production induced by extract of mouse keratinocyte.

### DISCLOSURE OF THE INVENTION

Dietary fibers are defined as the portion of foods indigestible by human digestive enzyme. There are two principal types of dietary fiber: soluble and insoluble, and it is the water soluble dietary fiber that is used in the present invention. The water soluble dietary fiber has high water holding capacity, and when water is added, it becomes a highly viscous liquid. Exemplary such water soluble dietary fibers include glucomannan, galactomannan, water soluble pectin, alginic acid, carageenan, fucoidan, and agar, and the preferred are glucomannan and galactomannan in view of the effectiveness.

Hydrolysate of water soluble dietary fiber may be a hydrolysate produced by hydrolyzing the water soluble dietary fiber as described above or a material containing such water soluble dietary fiber such as konjac or pectin by using an enzyme such as mannanase or pectinase, an acid, or an alkaline. Preferred among these are hydrolysates produced by using a mineral acid, and in particular, hydrochloric acid in view of handling convenience, reproducibility, economy, and the like.

The conditions used for the hydrolysis is not particularly limited, and any adequate conditions may be selected depending on the material used for the hydrolysis. For example, when an enzyme is used for the hydrolysis, the hydrolysis is typically conducted by dissolving the water soluble dietary fiber or the material containing such water soluble dietary fiber in water to a concentration of 0.1 to 5% by weight, and allowing the enzyme to react at the pH and temperature suitable for the enzyme selected typically for a period of about 30 minutes to 20 hours.

The hydrolysis using an acid or an alkaline may be conducted by dissolving the water soluble dietary fiber or the material containing such water soluble dietary fiber in water to a concentration of 0.1 to 5% by weight, and allowing the acid or the alkaline to react with the hydrolysate. For example, when hydrochloric acid is used for the hydrolysis, the hydrochloric acid may be used at a concentration of 0.05N to 1.5N, and preferably at 0.125N to 1N, and at a temperature of 35° C. to 90° C., and preferably 45° C. to 80° C., for a period of 30 minutes to 3 hours and preferably 1 hour to 2 hours.

The resulting hydrolysate may be used after optional purification by a method commonly used in the art such as centrifugation, column purification, and ultrafiltration, followed by drying.

Of the hydrolysate produced by the hydrolysis as described above, the preferred are those having a molecular weight in the range of several hundred daltons (D) to 1000 kilodaltons (KD), and more preferably, those having a molecular weight of several KD to several hundred KD in view of the favorable suppression of the IgE and ease of production into a preparation.

The thus obtained hydrolysate of water soluble dietary fiber exhibits action of suppressing production of the anti-IgE antibody as will be demonstrated bellow in the Examples, and therefore, it can be used in the form of a drug or a food as an agent for suppressing the IgE antibody or as an agent for improving allergic predisposition for a person who is susceptible for type I allergy such as atopic dermatitis, bronchial asthma, and allergic rhinitis.

When the agent for improving allergic predisposition or the agent for suppressing production of IgE antibody of the present invention is used as a drug, it may be administered either as a peroral preparation or as a parenteral preparation, and the dosage forms include solid preparation such as tablet, granules, capsule, and powder and liquid preparations such as syrup and elixir, and also, injection, suppository, spray, eye drops, and external medicine.

Such preparation may be prepared by using a pharmacologically acceptable carrier by the method commonly used for the hydrolysate of the water soluble dietary fiber. For example, an oral solid preparation may be produced by mixing the hydrolysate of the water soluble dietary fibers with an excipient and optional additives such as a binder, disintegrant, lubricant, colorant, corrective, or flavor, and producing a tablet, coated tablet, granules, powder, capsule, or the like by the method commonly used in the art. An injection may be prepared by using a diluting agent such as water, ethyl alcohol, macrogol, propylene glycol, or the like for the carrier, and mixing the carrier with an additive such as a pH adjusting agent or buffer, a stabilizer, and optionally with a solubilizer, pain reliever, topical anesthetic, or the like by the method commonly used in the art to thereby produce a subcutaneous, intramuscular, intravenous injection.

The agent for improving allergic predisposition and the agent for suppressing production of IgE antibody of the present invention may be used as a food such as functional health food. Exemplary such foods include confectionery such as biscuit, chocolate, candy, chewing gum, munchables, fries, ice cream, and jelly, bread, noodle, soybean products such as bean curd, yogurt, milk products such as butter, sauce, salad dressings, mayonnaise, seasonings such as rice seasoning, beverages such as fermented milk, juice, sport drink, and soup.

Such food may also have incorporated therein an additional ingredient which is acknowledged to be effective for allergy, and exemplary such ingredient include tea, perilla, tian cha, evening primrose, dandelion, persimmon leaves, mugwort, and oranges.

The agent for improving allergic predisposition or the agent for suppressing production of IgE antibody may be administered generally at a daily dose of about 30 mg to 30 g, and preferably about 100 mg to 3 g per adult in terms of the hydrolysate of the water soluble dietary fibers although the dose may vary depending on the conditions, weight, age, sex, and the like of the patient. And this daily dose may be administered either in a single dose or in 2 to 4 divided doses.

Next, the present invention is described in further detail by referring to the Examples.

### EXAMPLES

### Example 1

### Effect of Konjac Glucomannan on Suppressing IgE Antibody Production

(1) Production of Konjac Glucomannan Hydrolysate

20 mg of konjac glucomannan (purchased from Wako Pure Chemical Industries, Ltd.) was suspended in 2.4 mL of distilled water, and the suspension was shaken in a water bath at 50° C. for 2 hours to produce konjac gel. Hydrochloric acid was added to this gel to a final concentration of 0.2N, and the mixture was shaken for 2 hours. After returning to room temperature, sodium hydroxide was added to the mixture to neutralize hydrochloric acid, and 0.5 mL of 0.5M phosphate buffer (pH 6.5) was added to adjust the solution to a pH of 6.5. The resulting hydrolysate was centrifuged at 10000 rpm for 10 min to remove the insoluble content, and applied to Sephacryl S-200 column (1×45 cm, Amersham Bioscience) for fractionation by elution with 10 mM phosphate buffer (pH 6.5). The thus obtained fractions were evaluated for their total saccharide content by phenol sulfuric acid method, and it was found that the hydrolysate of the konjac glucomannan had been collected at an elution volume of 18 to 22.5 mL. The corresponding fractions were combined and dialyzed against phosphate buffer, and concentrated by an ultra-concentrator. The thus obtained hydrolysate was evaluated filtration for saccharide concentration by phenol sulfuric acid method.

(2) Effect of Suppressing IgE Antibody Production of Konjac Glucomannan in an In Vitro Antibody Production System

Spleen from a Balb/c mouse (8 week old, male) was dispersed in ISCOV medium to produce a cell suspension, and lymphocyte fractions were collected by density gradient centrifugation using Lympholite-M (CedarLane Laboratories). The thus prepared lymphocytes were adjusted to 2×10⁶/mL with ISCOV medium containing IL-4 (R&D; final concentration, 100 ng/mL), anti-CD-40 antibody (Serotec; final concentration, 200 ng/mL), and 2-mercaptoethanol (final concentration, 50 nM), and the suspension was poured to the wells of a 96-well microplate at 180 μL/well. To the well were also added 10 μL of PBS(−) and 10 μL of the hydrolysate of konjac glucomannan, and the cells were cultivated in a carbon dioxide incubator for 7 days. After the cultivation, the culture supernatant was recovered from the well, and the concentration of the antibody produced was measured. IgE was measured by using Mouse IgE Quantitative ELISA kit (Bethyl) in accordance with the instruction of the manufacturer.

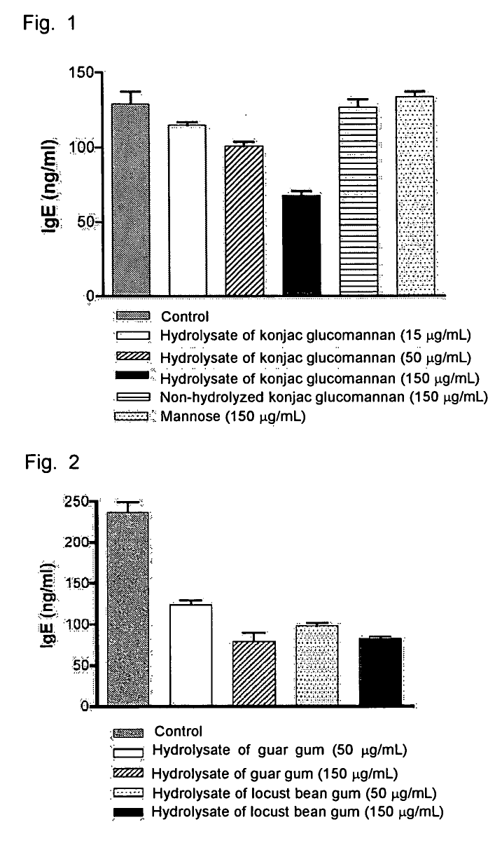

As shown in FIG. 1, the effect of suppressing the IgE production was not found for non-hydrolyzed konjac glucomannan and mannose which is one of the saccharides constituting the konjac glucomannan. In contrast, in the culture containing the hydrolysate of the konjac glucomannan at a final concentration of 15, 30, or 150 μg/mL, the amount of IgE produced in the culture decreased in a concentration dependent manner compared to the control, and the effect of suppressing the IgE production was most significant at 150 μg/mL. These results indicates the situation that the effect of suppressing the IgE antibody production is expressed only after digestion of the dietary fiber and reduction of the molecular weight by enteric bacteria, and the dietary fiber which has been preliminarily hydrolyzed for reduction of the molecular weight is expected to show an improved absorption, immediate action, and high effectiveness.

### Example 2

### Effect of Galactomannan on Suppressing IgE Antibody Production

(1) Production of Galactomannan Hydrolysate

20 mg of guar gum or locust bean gum (purchased from Sigma) was suspended in 2.4 mL of distilled water, and the suspension was shaken in a water bath at 50° C. for 3 hours to produce gel. Hydrochloric acid was added to this gel to a final concentration of 0.2N, and the mixture was shaken for 2 hours. After returning to room temperature, sodium hydroxide was added to the mixture to neutralize hydrochloric acid, and 0.5 mL of 0.5M phosphate buffer (pH 6.5) was added to adjust the solution to a pH of 6.5. The resulting hydrolysate was centrifuged at 10000 rpm for 10 min to remove the insoluble content, and applied to Sephacryl S-200 column (1×45 cm) for fractionation by elution with 10 mM phosphate buffer (pH 6.5). It was then found that the hydrolysate the galactomannan had been had been collected at an elution volume of 18 to 22.5 mL. The corresponding fractions were dialyzed against phosphate buffer, and concentrated by an ultra-concentrator. The concentration of the thus obtained hydrolysate was evaluated by phenol sulfuric acid method.

(2) Effect of Galactomannan on Suppressing IgE Antibody Production of in an In Vitro Antibody Production System

Spleen from a Balb/c mouse (8 week old, male) was dispersed in ISCOV medium to produce a cell suspension, and lymphocyte fractions were collected by density gradient centrifugation using Lympholite-M (CedarLane Laboratories). The thus prepared lymphocytes were adjusted to 2×10⁶/mL with ISCOV medium containing IL-4 (R&D; final concentration, 100 ng/mL), anti-CD-40 antibody (Serotec; final concentration, 200 ng/mL), and 2-mercaptoethanol (final concentration, 50 nM), and the suspension was poured to the wells of a 96-well microplate at 180 μL/well. To the well were also added 10 μL of PBS(−) and 10 μL of the hydrolysate of konjac glucomannan, and the cells were cultivated in a carbon dioxide incubator for 7 days. After the cultivation, the culture supernatant was recovered from the well, and the concentration of the IgE antibody produced was measured.

In the cultivation having the hydrolysate of the galactomannan added to a final concentration of 50 and 150 μg/mL, the IgE produced in the medium was significantly less than that of the control (FIG. 2).

### Example 3

### Molecular Weight Distribution and Effect of Suppressing IgE Antibody Production of Konjac Glucomannan

(1) Production of Konjac Glucomannan Hydrolysate

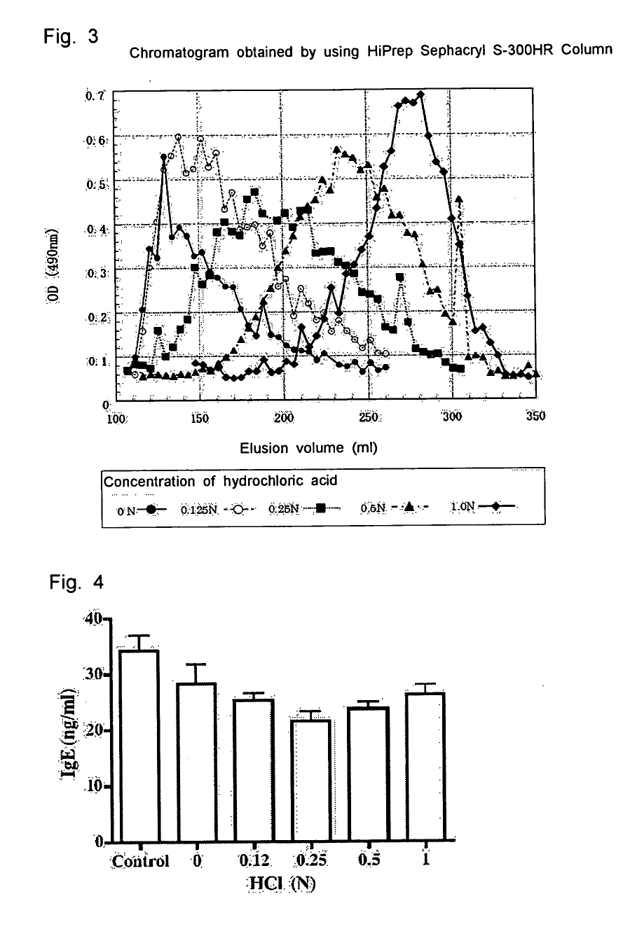

20 mg of konjac glucomannan (purchased from Wako Pure Chemical Industries, Ltd.) was suspended in 2 mL of distilled water, and the suspension was shaken in a water bath at 75° C. for 1 hour to produce konjac gel. Hydrochloric acid was added to this gel to a final concentration of 0.2N, 1.0N, 0.5N, 0.25N, and 0.125N, respectively, and the mixture was shaken for 1 hour. After returning to room temperature, sodium hydroxide was added to the mixture to neutralize hydrochloric acid, and 0.5 mL of 0.5M phosphate buffer (pH 6.5) was added to adjust the solution to a pH of 6.5. The resulting hydrolysate was centrifuged at 10000 rpm for 10 min to remove the insoluble content, and applied to Sephacryl S-300HR column (2.6×60 cm, Amercham Bioscience) for fractionation by elution with 10 mM phosphate buffer (pH 6.5). The thus obtained fractions were evaluated for their total saccharide content by phenol sulfuric acid method, and it was found that the hydrolysate eluted at a position corresponding to a lower molecular weight when the concentration of the hydrochloric acid was higher (FIG. 3). The range of molecular weight fractionation in this gel filtration column corresponds to 10 KD to 1500 KD in terms of globular protein, and 2 KD to 400 KD in terms of dextran (Amersham Bioscience). The results obtained for the hydrolyzed konjac glucomannan molecules indicated that their fractionation profile is similar to that of the dextran, and that the hydrolysate of the konjac glucomannan having a broad molecular weight distribution in the range of about 1 KD to about 400 KD had been produced (FIG. 3). Fractions corresponding to each peak of the total saccharide concentration were respectively combined to prepare a series of hydrolysates respectively covering the molecular weight range of 1 KD to several hundred KD. The combined fractions were dialyzed against phosphate buffer, and concentrated by using an ultra-concentrator corresponding to the estimated molecular weight. The saccharide concentration of the thus obtained hydrolysate was evaluated by phenol sulfuric acid method.

(2) Effect of Suppressing IgE Antibody Production of Konjac Glucomannan in an In Vitro Antibody Production System

The hydrolysate of the konjac glucomannan was measured for the effect of suppressing IgE antibody production in an in vitro antibody production system by repeating the procedure of Example 1 (2) and Example 2 (2). The samples used were the hydrolysates of the konjac glucomannan produced in (1) each having different molecular weight. After 7 days of cultivation, the culture supernatant was collected from the well to measure the IgE concentration.

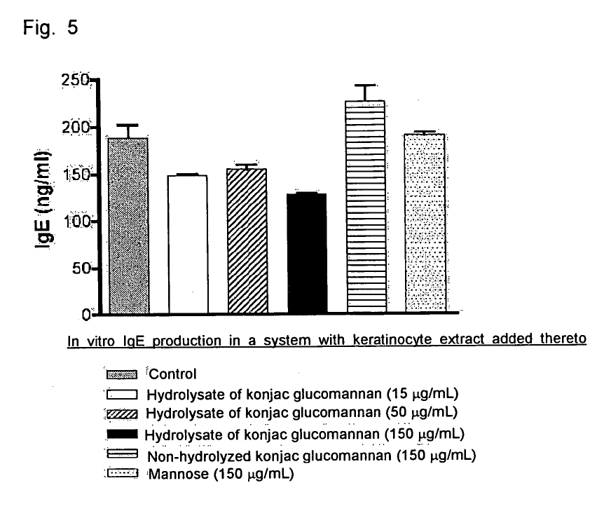

With regard to the effect of suppressing the IgE antibody production by the hydrolysates of the konjac glucomannan having different molecular weight, the effect increased with the decrease in the molecular weight approximately to the level of the hydrolysate produced at a hydrochloric acid concentration of 0.25N (namely, the molecular weight corresponding to about 60 KD in terms of globular protein). The effect of suppressing the IgE antibody production, however, somewhat decreased at the higher hydrochloric acid concentration (FIG. 4).

### Example 4

### Effects of the Hydrolysate of Konjac Glucomannan on IgE Production System by Keratinocyte Extract

Atopic dermatitis is associated with the vicious circle that itching of the skin induce scratching of the skin which in turn worsen of the skin condition and increase the itching. This phenomenon suggests the possibility that the keratinocyte destroyed by the itching may be a cause of the worsening of the conditions, and it has been revealed that IgE production is stimulated when extract of keratinocyte (PAM-212 cell) is administered to a Balb/c mouse. It has also been revealed that addition of PAM-212 cell extract to in vitro IgE production system produced by using splenocyte of a Balb/c mouse markedly enhances IgE production (Yamamoto, T., Kaneko, S. et al., Increase in serum IgE levels following injection of syngeneic keratinocyte extracts in BALB/c mice., Arch Dermatol Res 294:117-23 2002; Morimoto K. et al., Effect of IgE production enhancing factor from mouse keratinocyte strain on IgE class switch, Allergy 51(9, 10), 992 (Abst), 2002). Accordingly, the effect of suppressing IgE antibody production of the hydrolysate of konjac glucomannan on the IgE production enhancement was evaluated by using an in vitro IgE production system by keratinocyte extract.

(1) Effect of Suppressing IgE Antibody Production by Hydrolysate of Konjac Glucomannan in an In Vitro Antibody Production System

Spleen from a Balb/c mouse (8 week old, male) was loosened in ISCOV medium to produce a cell suspension, and lymphocyte fractions were collected by density gradient centrifugation using Lympholite-M (CedarLane Laboratories). The thus prepared lymphocytes were adjusted to 2×10⁶/mL with ISCOV medium containing IL-4 (R&D; final concentration, 100 ng/mL), anti-CD-40 antibody (Serotec; final concentration, 200 ng/mL), and 2-mercaptoethanol (final concentration, 50 nM), and the suspension was poured to the wells of a 96-well microplate at 180 μL/well. To the well were also added 10 μL of keratinocyte extract and 10 μL of the hydrolysate of konjac glucomannan, and the cells were cultivated in a carbon dioxide incubator for 7 days. The hydrolysate of konjac glucomannan was prepared by repeating the procedure of Example 1(1). After the cultivation, the culture supernatant was recovered from the well, and the concentration of the IgE antibody produced was measured.

Addition of the keratinocyte extract resulted in the increase of in vitro IgE production. In the cultures where the hydrolysate of konjac glucomannan was added to a final concentration of 15, 30, and 150 μg/mL, increase in the IgE production was clearly suppressed as in the case of Example 1(2), whereas such suppression was not at all noted in the culture having non-hydrolyzed konjac glucomannan added thereto (FIG. 4).

1. An agent for improving allergic predisposition comprising a hydrolysate of water-soluble dietary fibers as its effective component.

2. An agent for suppressing production of IgE antibody comprising a hydrolysate of water-soluble dietary fibers as its effective component.

3. The agent for suppressing production of IgE antibody according to claim 2 wherein the water-soluble dietary fibers is glucomannan or galactomannan.

4. A food for improving allergic predisposition comprising hydrolysate of water-soluble dietary fiber.

5. Use of a hydrolysate of water-soluble dietary fibers for producing an agent for improving allergic predisposition.

6. Use of a hydrolysate of water-soluble dietary fibers for producing an agent for suppressing production of IgE antibody.

7. A method for improving allergic predisposition comprising administering a hydrolysate of water-soluble dietary fiber.

8. A method for suppressing production of IgE antibody comprising administering a hydrolysate of water-soluble dietary fiber.
